(19) United States
(12) Patent Application Publication
(10) Pub. No.: US 2025/0244306 A1
(43) Pub. Date: Jul. 31, 2025

(54) Method and System for Analyzing Bimetallic Isotope Source of Cd/Pb Composite Pollution in Soil

(71) Applicant: Institute of Geochemistry, Chinese Academy of Sciences, Guiyang City (CN)

(72) Inventors: Yafei Xia, Guiyang City (CN); Chengshuai Liu, Guiyang City (CN); Ting Gao, Guiyang City (CN); Yuhui Liu, Guiyang City (CN)

(21) Appl. No.: 18/705,125

(22) PCT Filed: Oct. 25, 2022
(86) PCT No.: PCT/CN2022/127186

§ 371 Date: Apr. 26, 2024

(30) Foreign Application Priority Data
Oct. 28, 2021 (CN) 202111265253.8

(51) Int. Cl. G01N 33/24; H01J 49/10

(52) U.S. Cl. CPC G01N 33/24; H01J 49/105

(57) ABSTRACT

The present invention discloses a method for analyzing sources of Cd/Pb combined polluted farmland soil using bimetallic isotopes (Cd—Pb), comprising the following steps: collecting respectively a soil sample and risk source samples through a sample collection device; determining respectively Cd isotope ratio and Pb isotope ratio of the soil sample and the risk source samples; plotting by using the Cd isotope ratio and the Pb isotope ratio of the soil sample and the risk source samples as coordinates, to obtain a projection plot of the isotope ratios; recognizing a pollution end-member polluting farmland soil through the projection plot of the isotope ratios to obtain a recognition result for the pollution end-member, and then confirming the pollution end-member; and calculating a relative contribution rate of the pollution end-member, to obtain an analysis result for the source of isotopes. The present invention uses the two metal isotopes of Cd—Pb to mutually restrict and confirm each other to solve the problem of poor analytical accuracy caused by similar or overlapping isotope values of samples and isotope fractionation, and realizes accurate recognition and quantitative analysis for the source of heavy metals Cd/Pb pollution of farmland soil.

### TECHNICAL FIELD

The present invention relates to a research field of prevention and control for pollution of soil by heavy metals, particularly relates to a method and a system for analyzing sources of bimetallic isotopes of Cd/Pb combined pollution of soil.

### BACKGROUND

Heavy metals, Cd and Pb are globally recognized toxic metal elements. The migration of Cd and Pb in soil is challenging, and they possess characteristics such as high toxicity, resistance to degradation, and the ability to biomagnify through food chains and webs. These factors pose a significant threat to human health. Therefore, Cd/Pb combined pollution of soil has a certain particularity, is a difficult point for governing pollution of soil by heavy metals, and at the same time, has always been a hot spot and a difficult research topic at home and abroad. With an increasing extent of Cd/Pb pollution of farmland soil, simply studying morphology, types and spatial distribution of heavy metal pollutants in soil has no longer met the existing needs for governing pollution of farmland soil. However, a targeted treatment for pollution of farmland soil by heavy metals could hardly be performed, and the effect of treatment is unsatisfactory, since an environmental medium of farmland soil is very complicated, it is more difficult to accurately recognize a source of pollution, and it is difficult to quantify a pollution contribution. Therefore, in governance work for Cd/Pb pollution of soil, it seems to be very urgent and necessary to develop a method capable of effectively determining the source of Cd/Pb pollution in farmland soil and quantifying the contribution rate of each pollution source.

At present, a vast majority of work for analyzing the source of Cd and Pb pollution in soil generally relies on extensive databases and mathematical statistical analysis. For example, factor analysis method, principal component analysis method, clustering methodology, enrichment factor method and the like methods may be employed; however, qualitative analysis for types of the source of heavy metals in soil can only be achieved by means of these methods. In addition, qualitative and quantitative analysis for the source can be achieved by means of chemical mass balance method, positive matrix factorization method and the like methods; however, these methods are based on massively and comprehensively collecting samples and cumbersome mathematical analysis, bringing out larger workload and more difficult in distinguishing a multicomponent system.

In recent years, an application of trace to the source for fingerprint characteristics of metal isotopes has brought new train of thought to the method for analyzing the source of heavy metals in soil, along with a continuous development of chemical analysis technique. Each substance in nature has its own unique “tag” of isotopic composition, therefore, the sources of mixed substances can be distinguished by these specific “tags” of the isotopic compositions of different substances. There are 8 isotopes of Cd: ¹⁰⁶Cd, ¹⁰⁸Cd, ¹¹⁰Cd, ¹¹¹Cd, ¹¹²Cd, ¹¹³Cd, ¹¹⁴Cd, and ¹¹⁶Cd. Pb exists in nature in the form of four isotopes: ²⁰⁴Pb, ²⁰⁶Pb, ²⁰⁷Pb and ²⁰⁸Pb, wherein ²⁰⁴Pb is the only primitive and stable isotope formed in the Big Bang, and ²⁰⁶Pb, ²⁰⁷Pb and ²⁰⁸Pb are radioactive decay products of ²³⁸U, ²³⁵U and ²³²Th, respectively. At present, there have been a small number of reports about using one of the Cd or Pb isotopes for tracing to the source of soil pollution by single metal isotopes, and the pollution source is usually analyzed by a ratio of a concentration of the metal or its reciprocal to that of the stable isotope of the metal. Cloquet et al. collected possible pollution sources such as surrounding soil, dust particles, and residual slag in the boiler etc. at a smeltery, determined the Cd isotopes, and plotted by the Cd isotope value and the reciprocal of the Cd concentration to preliminarily judge the source of pollution. It was concluded, through linear analysis, that the dust particles and waste slag were the main sources of Cd pollution in soil at this area. Liu et al. analyzed the sources of Pb in soil by means of field monitoring and analyzing the ratio of Pb isotopes in the soil-paddy rice system. The results showed that the sources of Pb in paddy soil were background soil, chemical fertilizer, atmospheric sedimentation and irrigation water.

However, geochemical processes such as adsorption, dissolution, redox reactions and biological processes in soil would lead to fractionation of Cd isotopes, which would blur the signal of Cd isotopes of the pollution source, thereby reducing an accuracy of the result for analyzing the source of single isotope of Cd.

### SUMMARY

In order to overcome shortcomings and deficiencies of the prior art, a primary objective of the present invention is to provide a method and a system for analyzing sources of bimetallic isotopes of Cd/Pb combined pollution of soil.

The first objective of the present invention is to provide a method for analyzing sources of bimetallic isotopes of Cd/Pb combined pollution of soil.

The second objective of the present invention is to provide a system for analyzing sources of bimetallic isotopes of Cd/Pb combined pollution of soil.

The first objective of the present invention is realized by the following technical solutions.

A method for analyzing sources of bimetallic isotopes of Cd/Pb combined pollution of soil comprises the following steps:
collecting respectively a soil sample and risk source samples through a sample collection device, to obtain the soil sample and the risk source samples respectively;
determining respectively Cd isotope ratio and Pb isotope ratio of the soil sample and the risk source samples, to obtain the Cd isotope ratio and the Pb isotope ratio of the soil sample and the Cd isotope ratio and the Pb isotope ratio of the risk source samples;
plotting by using the Cd isotope ratio and the Pb isotope ratio of the soil sample as coordinates, and plotting by using the Cd isotope ratio and the Pb isotope ratio of the risk source samples as coordinates, to obtain a projection plot of the isotope ratios;
recognizing a pollution end-member polluting farmland soil through the projection plot of the isotope ratios to obtain a recognition result for the pollution end-member, and then confirming the pollution end-member; and
calculating a relative contribution rate of the pollution end-member, to obtain an analysis result for the source of isotopes.

Further, the step of collecting the soil sample is particularly to collect the soil sample through the sample collection device to obtain the soil samples at different distances; collecting the risk source samples is particularly to collect different types of risk source samples, including the first type of risk source samples, the second type of risk source samples, the third type of risk source samples, the fourth type of risk source samples and the fifth type of risk source samples.

Further, the soil samples at different distances are surface soil at a depth of 0-20 cm in farmland at different distances.

Further, the step of collecting the soil sample is particularly to collect the soil sample through the sample collection device, to obtain soil samples in different orientations; collecting the risk source samples is particularly to collect different types of risk source samples, including the first type of risk source samples, the second type of risk source samples, the third type of risk source samples, the fourth type of risk source samples and the fifth type of risk source samples.

Further, the Cd isotope ratio is expressed as δ^{114/110}Cd, and the Pb isotope ratio is expressed as ²⁰⁸Pb/²⁰⁰Pb and ²⁰⁶Pb/²⁰⁷Pb.

Further, the step of determining the Cd isotope ratios and the Pb isotope ratios the soil sample and the risk source samples is particularly as follows.

Determining Cd stable isotope ratio comprises the following steps: loading 2.8 mL of AG MP-IM (with 100-200 meshes) resin into a separation column, firstly washing the resin with 10 mL of 3.5 N HNO₃, 2 N HCL+8 N HF and 6 N HCl; adding ultrapure water to adjust the resin to be neutral, then respectively removing a matrix element of the sample with 10 mL of 2 N HCl, removing Mo with 10 mL of 1 N HCl, removing Pb with 20 mL of 0.3 N HCl, removing Zn with 20 mL of 0.06 N HCl, removing Sn with 10 mL of 0.012 N HCl, finally eluting and collecting Cd with 20 mL of 0.0012 N HCl; evaporating a collected pure Cd solution to dryness and then dissolving it into 3% HNO₃ for determination, finishing the determination by adopting a multi-collector inductively coupled plasma mass spectrometer (Neptune Plus MC-ICP-MS), and correcting mass discrimination by adopting a double spike method. The determined result of Cd stable isotope ratio is expressed as follows:

δ^{114/110}Cd=[(¹¹⁴Cd/¹¹⁰Cd)ₛₐₘₚₗₑ/(¹¹⁴Cd/¹¹⁰Cd)_{NIST 3108}−1]×1000,

wherein, (¹¹⁴Cd/¹¹⁰Cd)ₛₐₘₚₗₑ is a value of ¹¹⁴Cd/¹¹⁰Cd of the determined sample, and (¹¹⁴Cd/¹¹⁰Cd)_{NIST 3108} is a value of ¹¹⁴Cd/¹¹⁰Cd of a standard NIST 3108.

Determining Pb stable isotope ratio comprises the following steps: loading 1.5 mL of AG1-X8 (with 100-200 meshes) resin into a separation column, firstly washing alternately the resin with 6 N HCl and MQ for three times; then removing an impurity element of the sample with 1.5 mL of 1 N HBr and 1.5 mL of 2 N HCl respectively, finally eluting Pb with 1.5 mL of 6 N HCl and collecting a pure Pb solution; evaporating the collected pure Pb solution to dryness and dissolving it into 3% HNO₃ for determination, finishing the determination by adopting the multi-collector inductively coupled plasma mass spectrometer (Neptune Plus MC-ICP-MS), and correcting mass discrimination of the instrument by adopting a standard Tl 997 with ²⁰⁵Tl/²⁰³Tl=2.3871 calibrated, as an internal standard.

Further, the step of plotting by using the Cd isotope ratio and the Pb isotope ratio of the soil sample as coordinates, and plotting by using the Cd isotope ratio and the Pb isotope ratio of the risk source samples as coordinates, to obtain a projection plot of the isotope ratios is particularly as follows: plotting by using the Cd isotope ratio of the soil sample as abscissa and the Pb isotope ratio of the soil sample as ordinate; and plotting by using the Cd isotope ratio of the risk source samples as abscissa and the Pb isotope ratio of the risk source samples as ordinate, to obtain the projection plot of the isotope ratios.

Further, the step of recognizing the pollution end-member polluting farmland soil through the projection plot of the isotope ratios to obtain a recognition result for the pollution end-member, and then confirming the pollution end-member, is particularly as follows: the projection points of the ratio of isotopes polluting farmland soil are within the range surrounded by the projection points of the isotope ratios for various pollution end-members, therefore, various risk sources that are close to and surround the projection points of the ratios for isotopes polluting farmland soil can be recognized as the pollution end-member.

Further, the step of calculating relative contribution rates of the a pollution end-member, to obtain the analysis result for the isotope source are particularly as follows: calculating the relative contribution rates of different pollution end-members to Cd and Pb in the polluted farmland soil, through a calculation formula for analyzing the source.

Further, the calculation formulas for analyzing the source are particularly as follows:

$${δ}^{114 / 110} C {d}_{soil} = {δ}^{114 / 110} C {d}_{A} {x}_{A} + {δ}^{114 / 110} C {d}_{B} {x}_{B} + {δ}^{114 / 110} C {d}_{C} {x}_{C} + {δ}^{114 / 110} C {d}_{D} {x}_{D} ,$$

$${( \frac{mPb}{n P b} )}_{soil} = {( \frac{m P b}{n P b} )}_{A} {x}_{A} + {( \frac{m P b}{n P b} )}_{B} {x}_{B} + {( \frac{m P b}{n P b} )}_{C} {x}_{C} + {( \frac{m P b}{n P b} )}_{D} {x}_{D} ,$$

$$1 = {x}_{A} + {x}_{B} + {x}_{C} + {x}_{D} ,$$

wherein, A, B, C and D respectively represent four pollution end-members; δ^{114/110}Cd is Cd isotope ratio; δ^{114/110}Cd_{A}, δ^{114/110}Cd_{B}, δ^{114/110}Cd_{C}and δ^{114/110}Cd_{D} respectively represent Cd isotope ratios of the four pollution end-members of A, B, C and D; and

$${( \frac{m P b}{n P b} )}_{soil}$$

represents Pb isotope ratio of the polluted soil, and

$${( \frac{m P b}{n P b} )}_{A} , {( \frac{m P b}{n P b} )}_{B} , {( \frac{m P b}{n P b} )}_{C} and {( \frac{m P b}{n P b} )}_{D}$$

respectively represent Pb isotope ratios of the four pollution end-members of A, B, C and D. When m=208 and n=206, or when m=206 and n=207, x_{A}, x_{B}; x_{C} and x_{D} respectively represent the relative contribution rates of the four pollution end-members of A, B, C and D.

The second objective of the present invention is realized by the following technical solution.

A system for analyzing sources of bimetallic isotopes of Cd/Pb combined pollution in farmland soil comprises:
a sample collection module for collecting a soil sample and risk source samples;
a sample determination module for determining Cd isotope ratio and Pb isotope ratio of the soil sample and the risk source samples, to obtain the Cd isotope ratio and the Pb isotope ratio of the soil sample, and the Cd isotope ratio and the Pb isotope ratio of the risk source samples;
a plotting module for plotting by using the Cd isotope ratio and the Pb isotope ratio of the soil sample as coordinates, and plotting by using the Cd isotope ratio and the Pb isotope ratio of the risk source samples as coordinates, to obtain a projection plot of the isotope ratios;
a recognition and confirmation module for recognizing a pollution end-member polluting farmland soil by using the projection plot of the isotope ratios to obtain a recognition result for the pollution end-member, and then confirming the pollution end-member;
a calculation module of relative contribution rate for calculating a relative contribution rate of the pollution end-member, to obtain an analysis result for the source of isotopes; and
an output module of result for outputting a final analysis result for the source of isotopes.

Compared with the prior art, the present invention has the following advantages and beneficial effects.

1. The present invention relies on plots of the isotope ratios of Cd and Pb elements in farmland soil and pollution source samples, and can accurately recognize the Cd/Pb pollution end-member in farmland soil.

2. The present invention relies on the isotopic characteristics of Cd and Pb elements in farmland soil and pollution source samples, and can accurately and quantitatively analyze the source of Cd/Pb pollution in farmland soil, and determine the quantitative contribution rate of different pollution sources to Cd/Pb pollution of soil.

3. The present invention, through determining the Cd/Pb isotopes in soil and pollution source, has developed a method for tracing to the source of heavy metals polluting soil by relying on two-isotope fingerprint technique, which can use the two metal isotopes to mutually restrict and confirm each other. Compared with a traditional method such as multivariate statistics, single isotope fingerprint and so on, the contribution rates of different pollution source can be more accurately ascertained, the analysis results are more objective and accurate, and the traceability effect to the pollution source is better.

### DESCRIPTION OF DRAWINGS

FIG. 1 shows a flowchart of a method for analyzing sources of bimetallic isotopes of Cd/Pb combined pollution of farmland soil of the present invention;

FIG. 2 shows a chart for recognizing the pollution end-member of farmland soil in Example 1 of the present invention;

FIG. 3 shows a chart for recognizing the pollution end-member of farmland soil in Example 2 of the present invention;

FIG. 4 shows a structural block diagram for a system for analyzing sources of bimetallic isotopes of Cd/Pb combined pollution of farmland soil in the present invention.

### DETAILED DESCRIPTION OF EMBODIMENTS

The present invention will be further described below in detail in combination with Examples and the drawings, but embodiments of the present invention are not limited thereto.

### Example 1

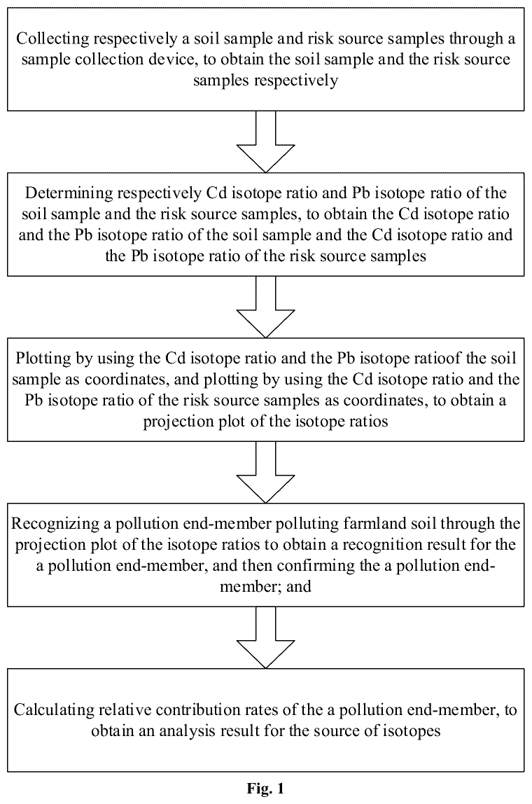

A method for analyzing sources of bimetallic isotopes of Cd/Pb combined pollution of farmland soil was shown as FIG. 1. It comprised the following steps:
collecting respectively a soil sample and risk source samples through a sample collection device, to obtain the soil sample and the risk source samples respectively;
determining respectively Cd isotope ratio and Pb isotope ratio of the soil sample and the risk source samples, to obtain the Cd isotope ratio and the Pb isotope ratio of the soil sample, and the Cd isotope ratio and the Pb isotope ratio of the risk source samples;
plotting by using the Cd isotope ratio and the Pb isotope ratio of the soil sample as coordinates, and plotting by using the Cd isotope ratio and the Pb isotope ratio of the risk source samples as coordinates, to obtain a projection plot of the isotope ratios;
recognizing the pollution end-member polluting farmland soil through the projection plot of the isotope ratios to obtain a recognition result for the pollution end-member, and then confirming the pollution end-member; and
calculating relative contribution rates of the pollution end-member, to obtain a analysis result for the source of isotopes.

Analysis of source of Cd/Pb combined pollution of farmland soil was performed by applying bimetallic isotopes of Cd/Pb, particularly as follows.

Farmland soil affected by a certain large-scaled lead-zinc mine field at a karst area in Guizhou province, China was selected as the implementation area. According to the pollution characteristics of the implementation area, surface soil samples at a depth of 0-20 cm were collected in the paddy field at distances of 200 meters, 1000 meters and 2000 meters away from the mine, respectively numbered as R1, R2 and R3. At the same time, risk source samples were collected, including tailings, mine dust, parent material, chemical fertilizer, and background soil. I.e., a soil sample at a first distance, a soil sample at a second distance, and a soil sample at a third distance were included. The soil sample at the first distance was surface soil at a depth of 0-20 cm in the farmland with the distance of 200 meters, the soil sample at the second distance was surface soil at a depth of 0-20 cm in the farmland with the distance of 1000 meters, and the soil sample at the third distance was surface soil at a depth of 0-20 cm in the farmland with the distance of 2000 meters. The first type of risk source samples were the tailings, the second type of risk source samples were the mine dust, the third type of risk source sample were the parent materials, the fourth type of risk source samples were the chemical fertilizers, and the fifth type of risk source samples were the background soils.

An isolation for the elements of Cd and Pb was respectively performed on the polluted farmland soil samples and the polluted risk source samples, and a purification treatment was carried out. Preferably, the Cd isotope ratio and the Pb isotope ratio were determined by adopting a multi-collector inductively coupled plasma mass spectrometer. The results for determination were shown as Table 1.

TABLE 1
The Cd isotope ratio and the Pb isotope ratio of the polluted
soil and the potential risk source samples in Example 1

| | δ^{114/110}Cd (‰) | ²⁰⁸Pb/²⁰⁶Pb | ²⁰⁶Pb/²⁰⁷Pb |
|---|---|---|---|
| chemical fertilizer | −0.3280 | 1.9612 | 1.2472 |
| background soil | −0.8172 | 2.0814 | 1.1757 |
| tailings | −0.4349 | 2.0820 | 1.1757 |
| parent material | −0.2350 | 2.0132 | 1.2117 |
| mine dust | −0.1961 | 2.1089 | 1.1574 |
| R1 | −0.2670 | 2.1067 | 1.1598 |
| R2 | −0.3417 | 2.1029 | 1.1619 |
| R3 | −0.5189 | 2.0879 | 1.1767 |

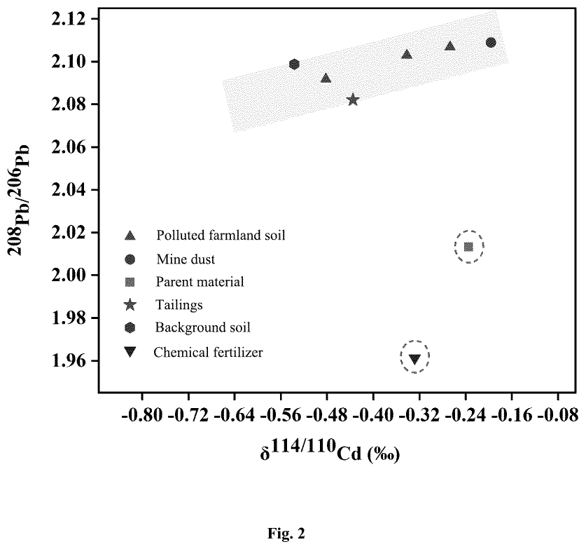

For plotting, the δ^{114/110}Cd of the farmland soil sample and the potential risk source sample was used as abscissa, and the ²⁰⁸Pb/²⁰⁶Pb of the same was used as ordinate in the present example, as shown in FIG. 2. It could be clearly seen from the figure that the heavy metal pollution sources in the farmland soil were mine dust, background soil and tailings. Among the three pollution end-members, the pollution sources from mines including tailings and mine dust had a relatively more obvious pollution impact. However, the contribution of chemical fertilizer and parent material could be excluded. It was showed that the farmland soil from the target study area was significantly impacted by mine exploitation, while the impact of pollution source of agricultural activities was almost masked.

The results for recognizing the end-members described above showed that there were three a pollution end-member of the farmland soil, namely, mine dust (A), tailings (B), and background soil (C), respectively. The δ^{114/110}Cd and the ²⁰⁸Pb/²⁰⁶Pb of the polluted farmland soil (Rn, n=1, 2, or 3) and the pollution end-member were introduced into the calculation formulas for pollution contribution:

$${δ}^{114 / 110} C {d}_{Rn} = {δ}^{114 / 110} C {d}_{A} {x}_{A} + {δ}^{114 / 110} C {d}_{B} {x}_{B} + {δ}^{114 / 110} C {d}_{C} {x}_{C} ,$$

$${( \frac{{}^{208} Pb}{{}^{206} Pb} )}_{Rn} = {( \frac{{}^{208} Pb}{{}^{206} Pb} )}_{A} {x}_{A} + {( \frac{{}^{208} Pb}{{}^{206} Pb} )}_{B} {x}_{B} + {( \frac{{}^{208} Pb}{{}^{206} Pb} )}_{C} {x}_{C} ,$$

$$1 = {x}_{A} + {x}_{B} + {x}_{C} ,$$

The obtained calculation results were shown in Table 2. R1 and R2 of the soil were closer to the mine, and mine dust contributed by 79.06% and 54.69%, respectively. The soil R3 was further away from the mine, the main pollution thereof came from background soil, and the contribution rate was 53.36%.

### Example 2

The present example was performed as the same way as that of Example 1, except for the following features.

Farmland soil affected by a certain large-scaled polymetallic mine field in South China was selected as the implementation area. According to the pollution characteristics of the implementation area, surface soil samples at a depth of 0-20 cm in four surrounding paddy fields with the mine field as a center were randomly collected, respectively numbered as P1, P2, P3 and P4. Risk source samples including mine wastewater precipitate, atmospheric sedimentation, parent material, chemical fertilizer, and background soil were collected at the same time. Namely, the four surrounding paddy fields included a paddy field at the first direction, a paddy field at the second direction, a paddy field at the third direction, and a paddy field at the fourth direction. The first type of risk source sample were mine wastewater precipitates, the second type of risk source samples were atmospheric sedimentation, the third type of risk source samples were the parent materials, the fourth type of risk source samples were chemical fertilizers, and the fifth type of risk source samples were background soils.

An isolation for the elements of Cd and Pb was respectively performed on the polluted farmland soil samples and the polluted risk source samples, and a purification treatment was carried out. Preferably, the Cd isotope ratio and the Pb isotope ratio were determined by adopting the multi-collector inductively coupled plasma mass spectrometer. The results for determination were shown as Table 3.

TABLE 3
The Cd isotope ratio and the Pb isotope ratio of the polluted
soil and the potential risk source samples in Example 2

| | δ^{114/110}Cd (‰) | ²⁰⁸Pb/²⁰⁶Pb | ²⁰⁶Pb/²⁰⁷Pb |
|---|---|---|---|
| mine wastewater precipitate | −0.0978 | 2.0891 | 1.1862 |
| chemical fertilizer | −0.0726 | 1.7948 | 0.7147 |
| parent material | −0.2650 | 2.0768 | 1.2523 |
| background soil | −0.0583 | 2.0771 | 1.1975 |
| atmospheric sedimentation | −0.2861 | 2.0946 | 1.1865 |
| P1 | −0.2510 | 2.0886 | 1.1879 |
| P2 | −0.1922 | 2.0886 | 1.1867 |
| P3 | −0.1986 | 2.0889 | 1.1865 |
| P4 | −0.2233 | 2.0890 | 1.1863 |

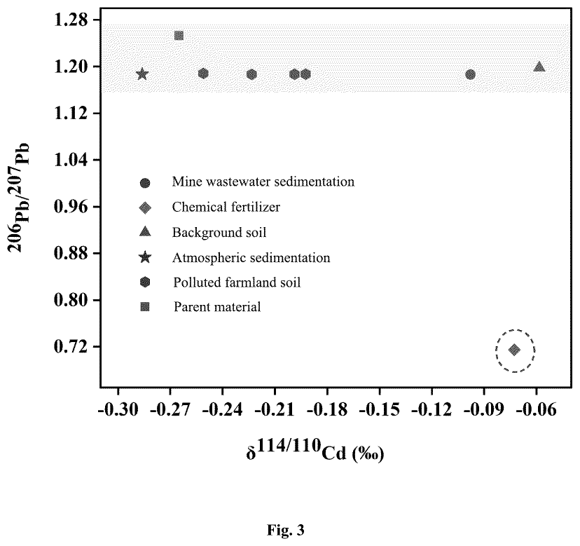

For plotting, the δ^{114/110}Cd of the farmland soil samples and the potential risk source samples was used as abscissa and the ²⁰⁶Pb/²⁰⁷Pb was used as ordinate in the present example, as shown in FIG. 3. It could be clearly seen from the figure that the pollution sources of heavy metals polluting farmland soil were four pollution end-members including mine wastewater precipitate, atmospheric sedimentation, parent material, and background soil. Compared to the impact from mining and metallurgical activities, the pollution contribution of chemical fertilizers from agricultural activities could be excluded.

A recognition result for the pollution end-member described above showed that there were four pollution end-members of farmland soil, namely, mine wastewater precipitate (A), atmospheric sedimentation (B), parent material (C), and background soil (D). The δ^{114/110}Cd and ²⁰⁶Pb/²⁰⁷Pb of the polluted farmland soil (Pn, n=1, 2, 3, or 4) and the pollution end-member were introduced into the calculation formulas for pollution contribution:

$${δ}^{114 / 110} C {d}_{Pn} = {δ}^{114 / 110} C {d}_{A} {x}_{A} + {δ}^{114 / 110} C {d}_{B} {x}_{B} + {δ}^{114 / 110} C {d}_{C} {x}_{C} + {δ}^{114 / 110} C {d}_{D} {x}_{D} ,$$

$${( \frac{{}^{208} Pb}{{}^{206} Pb} )}_{Pn} = {( \frac{{}^{208} Pb}{{}^{206} Pb} )}_{A} {x}_{A} + {( \frac{{}^{208} Pb}{{}^{206} Pb} )}_{B} {x}_{B} + {( \frac{{}^{208} Pb}{{}^{206} Pb} )}_{C} {x}_{C} + {( \frac{{}^{208} Pb}{{}^{206} Pb} )}_{D} {x}_{D} ,$$

$${( \frac{{}^{206} Pb}{{}^{207} Pb} )}_{Pn} = {( \frac{{}^{206} Pb}{{}^{207} Pb} )}_{A} {x}_{A} + {( \frac{{}^{206} Pb}{{}^{207} Pb} )}_{B} {x}_{B} + {( \frac{{}^{206} Pb}{{}^{207} Pb} )}_{C} {x}_{C} + {( \frac{{}^{206} Pb}{{}^{207} Pb} )}_{D} {x}_{D}$$

$$1 = {x}_{A} + {x}_{B} + {x}_{C} + {x}_{D} ,$$

The obtained results for calculation were shown as Table 4. At the red soil area in South China with stronger activity of heavy metals, the contribution of anthropogenic pollution by heavy metals was dominant, the contribution rate of mine wastewater precipitate and atmospheric sedimentation to the soil pollution of the four farmlands were 97% or more, while the contribution of natural sources including parent material and background soil was lower.

TABLE 4
Contribution amount (%) of the pollution
end-member in Example 2

| | mine | | | |
|---|---|---|---|---|
| | wastewater | atmospheric | parent | background |
| | precipitate | sedimentation | material | soil |
| DS-6 | 18.13 | 79.43 | 2.22 | 0.22 |
| DS-8 | 49.59 | 49.69 | 0.57 | 0.15 |
| DS-9 | 46.25 | 53.48 | 0.12 | 0.15 |
| DS-11 | 33.16 | 66.66 | 0.00 | 0.18 |

As mentioned above, according to the technical solutions of the present invention, the pollution end-member of farmland soil in different regions could be accurately recognized and the relative contribution rate could be calculated, by analyzing in parallel source of Cd/Pb combined pollution in soil by means of Cd/Pb isotopes. Two specific examples of the present invention were implemented at the karst area in Southwest China and at the red soil area in South China, with large differences in geological background and heavy metal activity. According to the projection plot of Cd—Pb isotopes, the pollution end-member of farmland soil were recognized, and the relative contribution rate of each pollution end-member was calculated. The above examples were better embodiments of the present invention, but embodiments of the present invention were not limited by the above examples.

### Example 3

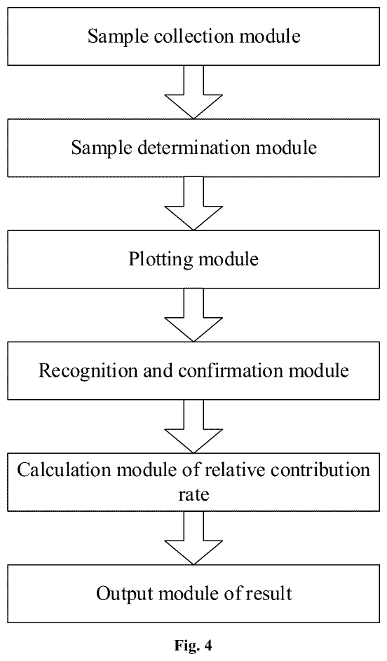

A system for analyzing sources of bimetallic isotopes of Cd/Pb combined pollution of farmland soil was as shown in FIG. 4, comprising:
a sample collection module for collecting a soil sample and risk source samples;
a sample determination module for determining Cd isotope ratio and Pb isotope ratio of the soil sample and the risk source samples, to obtain the Cd isotope ratio and the Pb isotope ratio of the soil sample, and the Cd isotope ratio and the Pb isotope ratio of the risk source samples;
a plotting module for ploting by using the Cd isotope ratio and the Pb isotope ratio of the soil sample as coordinates, and plotting by using the Cd isotope ratio and the Pb isotope ratio of the risk source samples as coordinates, to obtain a projection plot of the isotope ratios;
a recognition and confirmation module for recognizing the pollution end-member of the polluted farmland soil through the projection plot of the isotope ratios to obtain a recognition result for the pollution end-member, and then confirming the pollution end-member;
a calculation module of relative contribution rate for calculating a relative contribution rate of the pollution end-member, to obtain an analysis result for the source of isotopes; and
an output module of result for outputting a final analysis result for the source of isotopes.

The above-described Examples are preferred embodiments of the present invention, but embodiments of the present invention are not limited to the above-described Examples. Any other changes, modifications, substitutions, combinations, and simplifications made without departing from the spirit and the principle of the present invention should all be equivalent replacement modes and all be contained in the protection scope of the present invention.

1. A method for analyzing sources of bimetallic isotopes of Cd/Pb combined pollution of soil, characterized in that, it comprises the following steps:
collecting respectively a soil sample and risk source samples through a sample collection device, to obtain the soil sample and the risk source samples respectively;
determining respectively Cd isotope ratio and Pb isotope ratio of the soil sample and the risk source samples, to obtain the Cd isotope ratio and the Pb isotope ratio of the soil sample, and the Cd isotope ratio and the Pb isotope ratio of the risk source samples;
plotting by using the Cd isotope ratio and the Pb isotope ratio of the soil sample as coordinates, and plotting by using the Cd isotope ratio and the Pb isotope ratio of the risk source samples as coordinates, to obtain a projection plot of the isotope ratios;
recognizing a pollution end-member polluting farmland soil through the projection plot of the isotope ratios to obtain a recognition result for the pollution end-member, and then confirming the pollution end-member; and
calculating a relative contribution rate of the pollution end-member, to obtain an analysis result for the source of isotopes.

2. The method for analyzing the sources of bimetallic isotopes of Cd/Pb combined pollution of soil according to claim 1, characterized in that, the step of collecting the soil sample is particularly to collect the soil sample through the sample collection device to obtain the soil samples at different distances; the step of collecting the risk source samples is particularly to collect different types of risk source samples, including the first type of risk source samples, the second type of risk source samples, the third type of risk source samples, the fourth type of risk source samples and the fifth type of risk source samples.

3. The method for analyzing the sources of bimetallic isotopes of Cd/Pb combined pollution of soil according to claim 1, characterized in that, collecting the soil sample is particularly to collect in different orientations through the sample collection device; collecting the risk source samples is particularly to collect different types of risk source samples, including the first type of risk source samples, the second type of risk source samples, the third type of risk source samples, the fourth type of risk source samples and the fifth type of risk source samples.

4. The method for analyzing the sources of bimetallic isotopes of Cd/Pb combined pollution of soil according to claim 1, characterized in that, the Cd isotope ratio is expressed as δ^{114/110}Cd, and the Pb isotope ratio is expressed as ²⁰⁸Pb/²⁰⁶Pb and ²⁰⁶Pb/²⁰⁷Pb.

5. The method for analyzing the sources of bimetallic isotopes of Cd/Pb combined pollution of soil according to claim 4, characterized in that, the step of determining the Cd isotope ratios and the Pb isotope ratios the soil sample and the risk source samples is particularly as follows:
determining Cd stable isotope ratio: loading 2.8 mL of AG MP-1 M resin into a separation column, firstly washing the resin with 10 mL of 3.5 N HNO₃, 2 N HCL+8 N HF and 6 N HCl; adding ultrapure water to adjust the resin to be neutral, then removing matrix elements from the sample respectively with 10 mL of 2 N HCl, removing Mo with 10 mL of 1 N HCl, removing Pb with 20 mL of 0.3 N HCl, removing Zn with 20 mL of 0.06 N HCl, and removing Sn with 10 mL of 0.012 N HCl, finally eluting and collecting Cd with 20 mL of 0.0012 N HCl; evaporating the collected pure Cd solution to dryness and then dissolving it into 3% HNO₃ for determination, finishing the determination by adopting a multi-collector inductively coupled plasma mass spectrometer, correcting mass discrimination by means of a double spike method; with a result for the determined ratio of Cd stable isotope expressed as:
$${{{δ}^{114 / 110} C d = [ {(}^{114} Cd {/}^{110} C d )}_{s a m p l e} / {(}^{114} Cd {/}^{110} C d )}_{NIST 3108} - 1 ] \times 1000 ,$$
wherein, (¹¹⁴Cd/¹¹⁰Cd)ₛₐₘₚₗₑ is a value of ¹¹⁴Cd/¹¹⁰Cd of the determined sample, and (¹¹⁴Cd/¹¹⁰Cd) NIST ³¹⁰⁸ is a value of ¹¹⁴Cd/¹¹⁰Cd of a standard NIST ³¹⁰⁸; and
determining Pb stable isotope ratio: loading 1.5 mL of AG1-X8 resin into a separation column, firstly washing the resin alternately with 6 N HCl and MQ for three times; then removing impurity elements from the sample respectively with 1.5 mL of 1 N HBr, and 1.5 mL of 2 N HCl, finally eluting Pb with 1.5 mL of 6 N HCl and collecting it; evaporating the collected pure Pb solution to dryness and dissolving it into 3% HNO₃ for determination, finishing the determination by adopting a multi-collector inductively coupled plasma mass spectrometer, and correcting quality discrimination of the spectrometer by adopting a standard Tl 997 with ²⁰⁵Tl/²⁰³Tl=2.3871 calibrated, as an internal standard.

6. The method for analyzing the sources of bimetallic isotopes of Cd/Pb combined pollution of soil according to claim 1, characterized in that, the specific step of plotting by using the Cd isotope ratio and the Pb isotope ratio of the soil sample as coordinates, and plotting by using the Cd isotope ratio and the Pb isotope ratio of the risk source samples as coordinates to obtain the projection plot of the isotope ratios is particularly as follows: plotting by using the Cd isotope ratio of the soil sample as abscissa and the Pb isotope ratio of the soil sample as ordinate; and plotting by using the the Cd isotope ratio of the risk source samples as abscissa and the Pb isotope ratio of the risk source samples as ordinate, to obtain the projection plot of the isotope ratios having consistent abscissa and ordinate, with the abscissa being the Cd isotope and the ordinate being the Pb isotope.

7. The method for analyzing the sources of bimetallic isotopes of Cd/Pb combined pollution of soil according to claim 1, characterized in that, the step of recognizing the pollution end-member polluting farmland soil through the projection plot of the isotope ratios to obtain the recognition result for the pollution end-member, and then confirming the pollution end-member, is particularly as follows: the projection points of the ratio of isotopes polluting farmland soil are within the range surrounded by the projection points of the isotope ratios for various pollution end-members, therefore, various risk sources that are close to and surround the projection points of the ratios for isotopes polluting farmland soil can be recognized as the pollution end-member.

8. The method for analyzing the sources of bimetallic isotopes of Cd/Pb combined pollution of soil according to claim 1, characterized in that, the step of calculating the relative contribution rate of the pollution end-member, to obtain the analysis result for the source of isotopes is particularly as follows: calculating the relative contribution rates of different pollution end-members to Cd and Pb in polluted farmland soil through calculation formulas for analyzing the source.

9. The method for analyzing the sources of bimetallic isotopes of Cd/Pb combined pollution of soil according to claim 8, characterized in that, the calculation formulas for analyzing the source are particularly as follows:
$${δ}^{114 / 110} C {d}_{soil} = {δ}^{114 / 110} C {d}_{A} {x}_{A} + {δ}^{114 / 110} C {d}_{B} {x}_{B} + {δ}^{114 / 110} C {d}_{C} {x}_{C} + {δ}^{114 / 110} C {d}_{D} {x}_{D} ,$$
$${( \frac{mPb}{n P b} )}_{soil} = {( \frac{m P b}{n P b} )}_{A} {x}_{A} + {( \frac{m P b}{n P b} )}_{B} {x}_{B} + {( \frac{m P b}{n P b} )}_{C} {x}_{C} + {( \frac{m P b}{n P b} )}_{D} {x}_{D} ,$$
$$1 = {x}_{A} + {x}_{B} + {x}_{C} + {x}_{D} ,$$
wherein, A, B, C and D) respectively represent four pollution end-members; δ^{114/110}Cd is Cd isotope ratio; δ^{114/110}Cd_{A}, δ^{114/110}Cd_{B}, δ^{114/110}Cd_{C}and δ^{114/110}Cd_{D} respectively represent Cd isotope ratios of the four pollution end-members of A, B, C and D;
$${( \frac{m P b}{n P b} )}_{soil}$$
represents Pb isotope ratio of a polluted soil,
$${( \frac{m P b}{n P b} )}_{A} , {( \frac{m P b}{n P b} )}_{B} , {( \frac{m P b}{n P b} )}_{C} and {( \frac{m P b}{n P b} )}_{D}$$
respectively represent Pb isotope ratios of the four pollution end-members of A, B, C and D); when m=208 and n=206, or when m=206 and n=207, x_{A}, x_{B}, x_{C} and x_{D} respectively represent the contribution rates of the four pollution end-members of A, B, C and D.

10. A system for analyzing sources of bimetallic isotopes of Cd/Pb combined pollution of soil, characterized in that, it comprises:
a sample collection module for collecting a soil sample and risk source samples;
a sample determination module for determining Cd isotope ratio and Pb isotope ratio of the soil sample and the risk source samples, to obtain the Cd isotope ratio and the Pb isotope ratio of the soil sample and the Cd isotope ratio and the Pb isotope ratio of the risk source samples;
a plotting module for plotting by using the Cd isotope ratio and the Pb isotope ratio of the soil sample as coordinates, and plotting by using the Cd isotope ratio and the Pb isotope ratio of the risk source samples as coordinates, to obtain a projection plot of the isotope ratios;
a recognition and confirmation module for recognizing a pollution end-member polluting farmland soil through the projection plot of the isotope ratios to obtain a recognition result for the pollution end-member, and then confirming the pollution end-member;
a calculation module of relative contribution rate for calculating a relative contribution rate of the pollution end-member, to obtain an analysis result for the source of isotopes; and
an output module of result for outputting a final analysis result for the source of isotopes.
